(19) United States
(12) Patent Application Publication
(10) Pub. No.: US 2023/0248997 A1
(43) Pub. Date: Aug. 10, 2023

(54) ENERGY RADIATION TREATMENT METHOD AND SYSTEM SUPPORTING ENERGY RADIATION TREATMENT

(71) Applicant: OLYMPUS CORPORATION, Tokyo (JP)

(72) Inventor: Teppei TSURUTA, Tokyo (JP)

(73) Assignee: OLYMPUS CORPORATION, Tokyo (JP)

(21) Appl. No.: 18/105,272

(22) Filed: Feb. 3, 2023

Related U.S. Application Data
(60) Provisional application No. 63/307,821, filed on Feb. 8, 2022.

(51) Int. Cl. A61N 5/10

(52) U.S. Cl. CPC A61N 5/1039; A61N 5/1071; A61N 2005/1098

(57) ABSTRACT

An energy radiation treatment method comprises: administering a medicinal agent to an affected part; performing energy radiation with a predetermined energy on the affected part; and confirming therapeutic effects by the energy radiation on the affected part, wherein in the administering, a determination based on at least one ultrasound image based on ultrasound waves reflected from the affected part is performed.

### CROSS-REFERENCE TO RELATED APPLICATIONS

This application claims the benefit of priority to U.S. Provisional App. No. 63/307,821, filed Feb. 8, 2022, the entire contents of which are incorporated herein by reference.

### BACKGROUND

The present disclosure relates to an energy radiation treatment method for performing energy radiation treatment, and to a system supporting energy radiation treatment.

### DESCRIPTION OF RELATED ART

In recent years, energy radiation treatment such as photoimmunotherapy (PIT) has attracted attention. For example, in the photoimmunotherapy, a fluorescent marker specifically binding to cancer cells is used. The fluorescent marker administered into a human body binds to cancer cells. When near-infrared light having a predetermined wavelength is applied to the fluorescent marker, the cancer cells bound to the fluorescent marker are destroyed and go extinct.

In a case where the cancer cells are present on a surface of an organ or the like, a doctor can confirm a position and a size of the cancer cells by viewing a white light observation image such as an endoscope image. In addition, the doctor can confirm therapeutic effects of the photoimmunotherapy by viewing the white light observation image such as the endoscope image.

However, in a case where the cancer cells are present inside a parenchymal organ, the doctor cannot confirm a position and a size of the cancer cells even though the doctor views the white light observation image such as the endoscope image. Therefore, after an X-ray image is picked up in advance and a position and a size of cancer cells are confirmed, the doctor determines a method of administering a medicinal agent (for example, intravenous injection or local injection). After radiation of laser light, it is necessary for the doctor to pick up an X-ray image again and to confirm the therapeutic effects by the photoimmunotherapy.

### SUMMARY

An energy radiation treatment method comprising: administering a medicinal agent to an affected part; performing energy radiation with the predetermined energy on the affected part; and confirming therapeutic effects by the energy radiation on the affected part, wherein, in the administering, a determination based on at least one ultrasound image based on ultrasound waves reflected from the affected part is performed.

Further, a system supporting energy radiation treatment according to an embodiment comprising: an ultrasound endoscope including a channel, the ultrasound endoscope being configured to acquire an ultrasound image of an affected part, the channel allowing insertion of an energy radiation probe to perform energy radiation with predetermined energy on the affected part and being used to administer the medicinal agent to the affected part; and a processor comprising hardware, the processor being configured to determine a method of administering the medicinal agent to the affected part and a radiation condition of the energy radiation based on image comparison of the ultrasound image.

### BRIEF DESCRIPTION OF THE DRAWINGS

FIG. 1 is a block diagram illustrating an energy radiation treatment system according to a first embodiment;

FIG. 2 is a flowchart of an energy radiation treatment method according to the first embodiment;

FIG. 3 is an explanatory diagram of a subject;

FIG. 4 is an explanatory diagram schematically illustrating an example of an ultrasound image;

FIG. 5 is an explanatory diagram to explain administration of a medicinal agent and phototherapy using an ultrasound endoscope;

FIG. 6 is an explanatory diagram illustrating a state where a puncture needle is stuck into a pancreatic cyst;

FIG. 7 is an explanatory diagram illustrating a state where the puncture needle is stuck into a pancreatic duct;

FIG. 8 is an explanatory diagram illustrating a state where the puncture needle is stuck into a pancreatic parenchyma;

FIG. 9 is an explanatory diagram of confirming therapeutic effects by the energy radiation treatment on the affected part;

FIG. 10 is an explanatory diagram illustrating an image of an affected part in an ultrasound enhanced mode before radiation of therapeutic light;

FIG. 11 is an explanatory diagram illustrating the image of the affected part in the ultrasound enhanced mode after the radiation of the therapeutic light;

FIG. 12 is a block diagram illustrating a second embodiment;

FIG. 13 is an explanatory diagram illustrating a schematic configuration of a distal end part of an insertion portion of a duodenoscope;

FIG. 14 is an explanatory diagram to explain the energy radiation treatment method using the duodenoscope 41;

FIG. 15 is a block diagram illustrating a modification; and

FIG. 16 is a block diagram illustrating a third embodiment.

### DETAILED DESCRIPTION

Generally, in a case where cancer cells are present inside a parenchymal organ, acquisition of an X-ray image by an X-ray apparatus is performed not only to confirm a position and a size of the cancer cells before light radiation, but also to confirm therapeutic effects after light radiation. The therapeutic effects are confirmed not only immediately after the treatment but also on and after the next day of the treatment. Therefore, separately from an endoscope apparatus for treatment, it is necessary to use an apparatus for observation over the entire process from preparation of the treatment to confirmation of the therapeutic effects, which makes rapid treatment difficult.

According to embodiments described below, it is possible to solve such issues.

The embodiments are described in detail below with reference to the drawings.

### First Embodiment

In the present embodiment, in a case where a lesion part is treated using an apparatus performing energy radiation treatment, an ultrasound image is acquired by at least one of administration of a medicinal agent, placement of an energy radiation device and energy radiation, or confirmation of therapeutic effects, and each is performed with reference to the acquired ultrasound image. In the present embodiment, an example in which photoimmunotherapy (PIT) is performed by adopting a device generating near-infrared light as the energy radiation device is particularly described. The energy radiation device is not limited to a device generating light, other devices, such as a device radiating ultrasound waves or a neutron beam may be adopted. The present embodiment is similarly applicable to implementation of ultrasound treatment and neutron beam treatment, in addition to PIT.

### (Configuration)

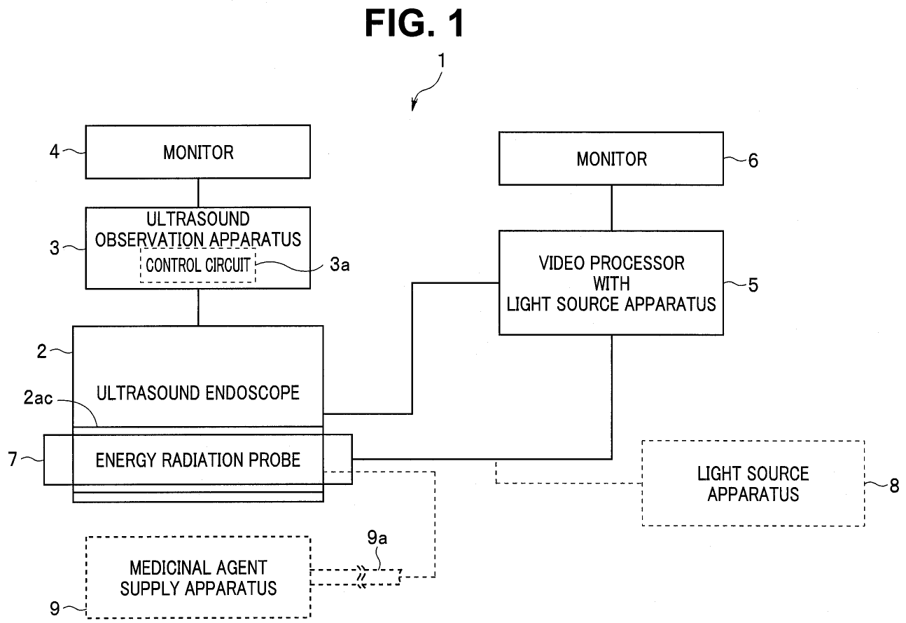

FIG. 1 is a block diagram illustrating an energy radiation treatment apparatus according to the first embodiment.

An energy radiation treatment apparatus 1 includes an ultrasound endoscope 2, an ultrasound observation apparatus 3, a video processor with light source apparatus 5, monitors 4 and 6, and an energy radiation probe 7 as the energy radiation device. In addition to the light source apparatus included in the video processor with light source apparatus 5, a light source apparatus 8 dedicated for the energy radiation probe 7 as the energy radiation device can be provided in some cases.

In a case where energy used by the energy radiation device such as the energy radiation probe 7 and energy used by an image observation device such as the ultrasound endoscope 2 are the same type, the energy can be supplied from a common supply source. In an example of FIG. 1, the video processor with light source apparatus 5 supplies light to the ultrasound endoscope 2, and also supplies light to the energy radiation probe 7. In a case where the energy radiation probe 7 generates ultrasound waves, output of the ultrasound observation apparatus 3 is provided not only to the ultrasound endoscope 2 but also to the energy radiation probe 7. In the case, when an output value, a frequency, and the like of an output wave are different between an energy radiation mode and an image observation mode, an output mode may be alternately switched between energy radiation and image observation, and reception information in the energy radiation mode may be superimposed on an image in the image observation mode.

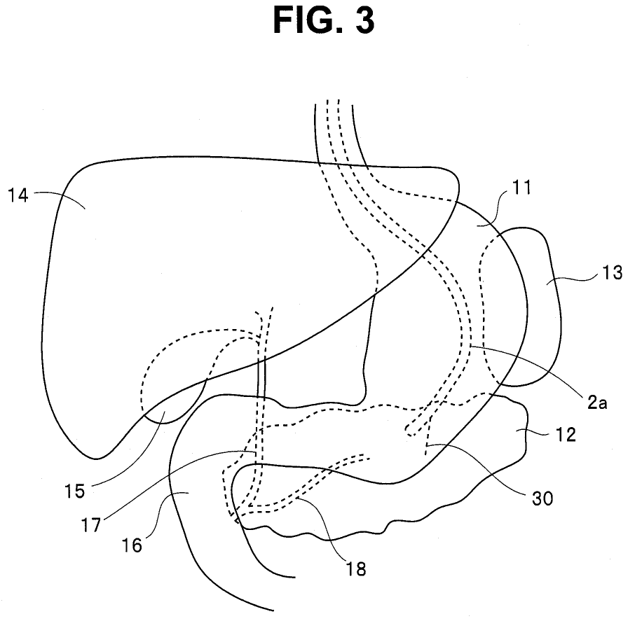

The ultrasound endoscope 2 includes an elongated insertion portion 2a (see FIG. 3) that has flexibility to be insertable into a body cavity, on a distal end side, and includes an unillustrated operation portion on a hand side. The ultrasound observation apparatus 3, the video processor with light source apparatus 5, the light source apparatus 8, and the like are connected through a cable extending from the operation portion.

A distal end of the insertion portion of the ultrasound endoscope 2 is bendable by operation of the operation portion. A distal end part of the insertion portion is provided with an ultrasound transducer that includes an unillustrated ultrasound oscillator generating ultrasound waves. The ultrasound waves from the ultrasound oscillator are radiated to a living body from the distal end of the ultrasound endoscope 2, reflected waves corresponding to characteristics of the living body are received by the ultrasound oscillator, and a reception signal (ultrasound signal) is accordingly acquired. When a plurality of ultrasound oscillators are arranged in, for example, a band shape (linear array), a ring shape (annular array), or a disc matrix (disc array), and are sequentially driven, it is possible to, for example, radially radiate the ultrasound waves and to acquire an ultrasound image of a predetermined range. Note that the ultrasound transducer may use a single ultrasound oscillator, and the ultrasound oscillator may be, for example, mechanically radially driven by a servo motor or the like, thereby acquiring the ultrasound image of the predetermined range.

The ultrasound transducer supplies the received ultrasound signal to the ultrasound observation apparatus 3. The ultrasound observation apparatus 3 generates an ultrasound image based on the received ultrasound signal by a well-known method. The ultrasound image generated by the ultrasound observation apparatus 3 is supplied to the monitor 4 and is displayed on a screen of the monitor 4.

The ultrasound endoscope 2 can also obtain an optical image in addition to the ultrasound image. The ultrasound endoscope 2 is supplied with illumination light from the video processor with light source apparatus 5, and irradiates the living body with the illumination light. The distal end part of the ultrasound endoscope 2 is provided with an unillustrated image pickup device such as a CCD, and reflected light from the living body is received by the image pickup device, and is converted into an image pickup signal. The image pickup signal from the image pickup device is supplied to the video processor with light source apparatus 5, and the video processor with light source apparatus 5 generates an endoscope image based on the image pickup signal. The video processor with light source apparatus 5 supplies the generated endoscope image to the monitor 6. As a result, the endoscope image based on the optical image is displayed on a screen of the monitor 6.

The insertion portion of the ultrasound endoscope 2 is provided with a treatment instrument channel 2ac, and the operation portion is provided with an opening portion communicating with the treatment instrument channel 2ac. The unillustrated hollow puncture needle and the energy radiation probe 7 such as a light radiation probe serving as the light radiation device can be inserted into the treatment instrument channel 2ac through the opening portion. The puncture needle and the energy radiation probe 7 inserted into the treatment instrument channel 2ac can be protruded from a treatment instrument opening at the distal end portion of the ultrasound endoscope 2. Further, the energy radiation probe 7 can be inserted into a needle tube of the puncture needle, and the energy radiation probe 7 inserted into the needle tube of the puncture needle can be protruded from the distal end portion of the ultrasound endoscope 2.

The energy radiation probe 7 is supplied with light from the video processor with light source apparatus 5 or the light source apparatus 8, and can irradiate inside of the living body with the light from the distal end of the ultrasound endoscope 2. In the present embodiment, an example in which a light radiation probe is used as the energy radiation probe 7 is described. In a case where a device radiating ultrasound waves or a neutron beam is used as the energy radiation probe 7, an ultrasound generation apparatus or a neutron beam generation apparatus is adopted in place of the light source apparatus 8.

Note that a medicinal agent supply apparatus 9 to inject a medicinal agent used for the energy radiation treatment is adopted in some cases. The medicinal agent supply apparatus 9 supplies the medicinal agent to a puncture needle 30 (see FIG. 3) inserted into the treatment instrument channel 2ac of the ultrasound endoscope 2, thereby enabling injection of the medicinal agent to an affected part.

### Treatment

In the present embodiment, as the energy radiation treatment, PIT is described as an example. In PIT, the following first to third steps are performed.

(A) First step ... including administration of medicinal agent
(B) Second step ... including placement of light radiation device and radiation
(C) Third step ... including confirmation of therapeutic effects
In the present embodiment, in the three steps (A), (B) and (C), the ultrasound image acquired by the ultrasound endoscope 2 is used.

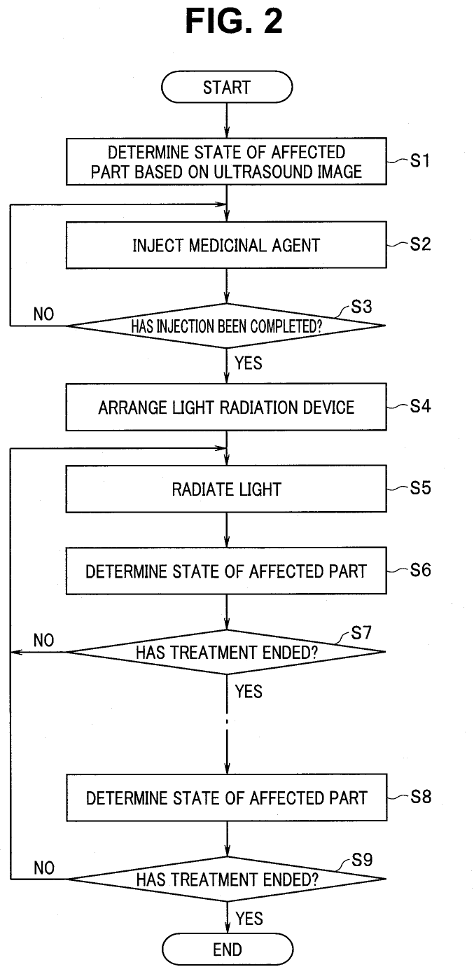

FIG. 2 is a flowchart to explain steps in the above-described method. In FIG. 2, steps S1 to S3 correspond to the first step, steps S4 and S5 correspond to the second step, and steps S6 to S9 correspond to the third step.

Taking pancreatic cancer as an example of a treatment target, the treatment method of PIT is described below.

### First Step

FIG. 3 to FIG. 8 are explanatory diagrams to explain the first to third steps. FIG. 3 schematically illustrates a state of puncture to a pancreas in the body. FIG. 3 illustrates a stomach 11, a pancreas 12, a spleen 13, a liver 14, a gallbladder 15, a duodenum 16, a bile duct 17, and a pancreatic duct 18 in a human body. FIG. 3 illustrates an insertion state of the ultrasound endoscope 2 in a case where the treatment target is the pancreas 12.

The first step includes a procedure to determine a method of administering the medicinal agent (medicinal solution). For the procedure, a doctor determines a state of the affected part based on the ultrasound image in S1 of FIG. 2. In other words, as illustrated in FIG. 3, the doctor orally introduces the ultrasound endoscope 2, inserts the distal end part of the insertion portion 2a of the ultrasound endoscope 2 illustrated by a dashed line into the stomach 11, and arranges the ultrasound oscillator provided at the distal end of the insertion portion 2a, at a position pressed with predetermined pressing force, on a stomach wall facing the pancreas 12. In the case, the ultrasound endoscope 2 is arranged such that an ultrasound emission surface of the ultrasound oscillator faces the pancreas. In the state, the doctor performs radiation of the ultrasound waves from an inner surface side on a wall part of the stomach 11. The doctor observes the affected part with cancer cells inside the pancreas adjacent to the stomach by using the ultrasound image acquired by the ultrasound endoscope 2. Note that, depending on a site of the pancreas, the doctor arranges the ultrasound endoscope 2 in the duodenum to observe the affected part in some cases. In the following, this is true of the second step and the third step.

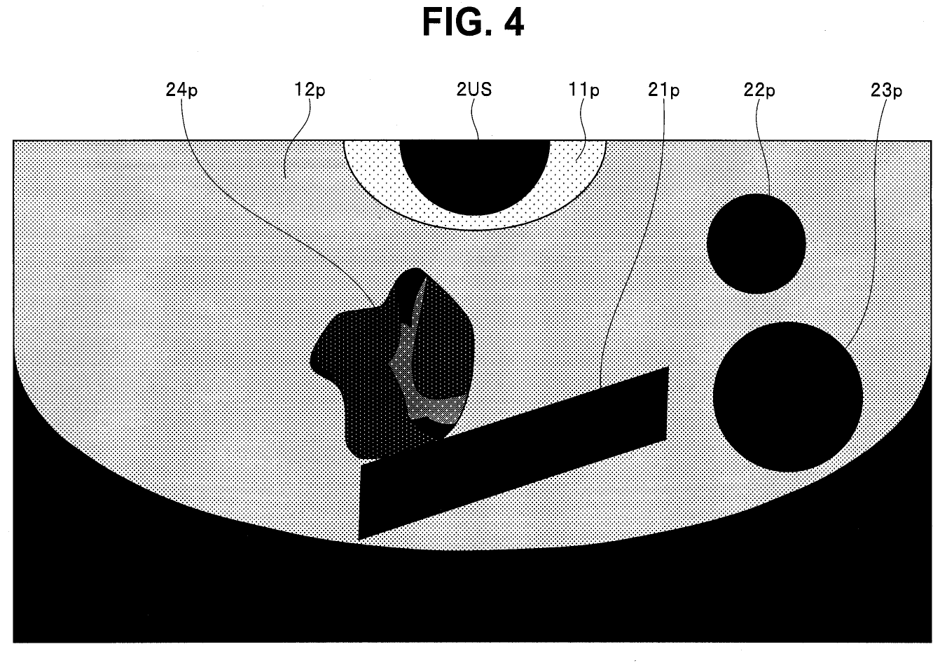

FIG. 4 is an explanatory diagram schematically illustrating an example of the ultrasound image. FIG. 4 illustrates an example of a convex ultrasound endoscope image.

In the ultrasound image in FIG. 4, an image part 2US having an arc shape at an upper end at a center in a right-left direction of the image is an image of the ultrasound transducer at the distal end of the ultrasound endoscope 2. A stomach wall image 11p can be acquired by pressing the distal end of the ultrasound endoscope 2 against the stomach wall of the stomach 11. A pancreas image 12p showing the pancreas 12 includes a pancreatic duct image 21p showing the pancreatic duct, a blood vessel image 22p showing a blood vessel, a cyst image 23p showing a cyst, and a tumor image 24p showing a tumor. The tumor image 24p is internally divided into regions different in gradation, and it is known that the inside of the actual typical pancreas cancer is drawn in a mottled pattern.

The doctor determines the method of administering the medicinal agent based on an observation result. As the method of administering the medicinal agent, for example, a method of administering the medicinal agent by intravenous injection and a method of administering the medicinal agent to the affected part or a periphery of the affected part by using the ultrasound endoscope 2 (hereinafter, referred to as direct injection) can be considered. In a case of the direct injection, the doctor also determines an injection position.

For example, the doctor determines the method of administering the medicinal agent based on a position of the tumor, an intervening substance (inhibitor (stroma) between cells) in administration of the medicinal agent, a state of the blood vessels, and the like. For example, in a case where a large number of blood vessels are present, the doctor selects the intravenous injection, and otherwise, the doctor selects the direct injection in some cases. Further, blood vessels are normally formed by the cancer, and density of the blood vessels is high in some cases. In the case, the doctor may select the method by the intravenous injection because the medicinal agent easily accumulates on the affected part through the blood vessels. When the cancer progresses, the blood vessels are destroyed and the density of the blood vessels becomes low. For example, in a case of pancreatic cancer, it is difficult to detect early cancer, and the blood vessels have been already destroyed when the pancreatic cancer is detected, in some cases. In such a case, the doctor may select the direct injection. After determining the injection method and the injection position of the medicinal agent, the doctor injects the medicinal agent (S2).

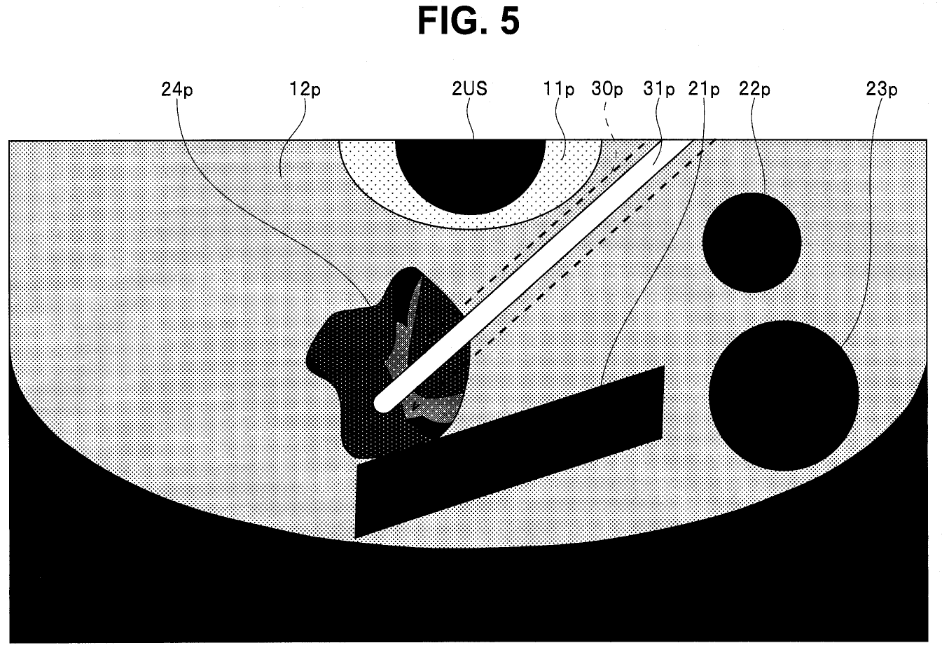

FIG. 5 is an explanatory diagram to explain administration of the medicinal agent in the first step and phototherapy in the second step using the ultrasound endoscope 2.

The doctor inserts the puncture needle 30 into the treatment instrument channel 2ac of the ultrasound endoscope 2, and protrudes the puncture needle 30 from the distal end of the ultrasound endoscope 2 as illustrated in FIG. 3. A distal end of the puncture needle 30 penetrates through the stomach wall and reaches the pancreas 12. The ultrasound endoscope 2 can be arranged such that the puncture needle 30 is positioned in a shooting range. FIG. 5 illustrates that a puncture needle image 30p (dashed line) showing the puncture needle 30 is included in the ultrasound image. The doctor arranges the puncture needle 30 at a desired position while viewing the puncture needle image 30p showing the puncture needle 30 on the ultrasound image. FIG. 5 illustrates that an image (hereinafter, referred to as a probe distal-end image) 31p of the distal end part of the energy radiation probe 7 inserted into the needle tube of the puncture needle 30 is acquired, but the energy radiation probe 7 is not inserted into the puncture needle 30 at a stage of the first step.

When determining that the distal end of the puncture needle has reached the affected part, the doctor injects the medicinal agent to the affected part. In other words, the doctor provides the medicinal agent into the needle tube of the puncture needle 30, and administers the medicinal agent to the target site (direct injection) from the distal end of the puncture needle 30. In the example of FIG. 5, the direct injection of the medicinal agent is performed in a state where the puncture needle 30 is arranged such that the distal end of the puncture needle 30 reaches the tumor shown in the tumor image 24p.

Thereafter, it is determined whether the injection of the medicinal agent has been completed (S3). For example, by using a photoacoustic image described below, a dispersion state of the medicinal agent, namely, an accumulation degree of the medicinal agent on the target site may be determined. In a case where it is determined that accumulation of the medicinal agent on the affected part is insufficient, the injection of the medicinal agent is continued. In a case where it is determined that accumulation of the medicinal agent on the affected part is sufficient, the injection of the medicinal agent is terminated.

Note that when the medicinal agent is administered in the first step, tissue fluid may be removed. When the medicinal agent is administered after the tissue fluid is removed, the medicinal agent may advantageously easily penetrate into the affected part. Further, in place of the tissue fluid, liquid or gas as substitute for the tissue fluid, for example, physiological saline, may be supplied.

### Second Step

In the second step, the energy radiation probe can be placed by a procedure similar to the procedure in the direct injection in the first step. For example, after the energy radiation probe 7 is inserted into the needle tube of the puncture needle 30 and it is confirmed that the energy radiation probe 7 has reached the vicinity of the affected part in the ultrasound image, therapeutic light may be emitted. Note that, before execution of the second step, a predetermined waiting time period until the medicinal agent is sufficiently distributed to the affected part after the medicinal agent is administered in the first step can be provided.

In other words, after the injection of the medicinal agent by the intravenous injection or the direct injection is completed, the doctor arranges the energy radiation probe 7 as the light radiation device at a position where energy radiation can be performed on the medicinal agent accumulated on the target site (S4). The example in FIG. 5 illustrates a state where, after the injection of the medicinal agent, the energy radiation probe 7 is inserted into the needle tube of the puncture needle 30 while the puncture needle 30 is placed, the distal end of the energy radiation probe 7 is protruded from the distal end of the puncture needle 30 as shown in the probe distal-end image 31p, and the energy radiation probe 7 is placed at the position where the tumor shown in the tumor image 24p can be irradiated with the light from the energy radiation probe 7.

The doctor causes the energy radiation probe 7 to irradiate the target site with the therapeutic light supplied from the video processor with light source apparatus 5 or the light source apparatus 8 in a state where the energy radiation probe 7 is arranged at the desired position. In the example of FIG. 5, a tumor part shown in the tumor image 24p is irradiated with the light. The medicinal agent reacting to the light is accumulated on the tumor part, and the medicinal agent reacts to irradiation with the light. As a result, the cancer cells go extinct.

The example in which the energy radiation probe 7 is inserted into the needle tube of the puncture needle 30 and the distal end of the energy radiation probe 7 is placed on the affected part is described. Alternatively, the puncture needle 30 may be removed from the treatment instrument channel 2ac, the energy radiation probe 7 may be directly inserted into the treatment instrument channel 2ac in place of the puncture needle 30, and the energy radiation probe 7 may be placed on the affected part through a fistula formed by the puncture needle 30. Further, the fistula may be dilated to facilitate insertion of the energy radiation probe 7. In a case of using the fistula, the energy radiation probe 7 having a relatively large diameter is adoptable, which may make it possible to perform light radiation with sufficient power.

To place the energy radiation probe 7 on the affected part, a treatment instrument other than the puncture needle 30 may be used. For example, a guide wire may be inserted into the puncture needle 30, the puncture needle 30 may be drawn out while only the guide wire is left in the body, and then the energy radiation probe 7 may be placed on a target site by using the guide wire.

Depending on a site to be treated, the energy radiation device may interfere with natural excretion of the tissue fluid. Further, the light radiation may be influenced by the tissue fluid because a transmittance is reduced and a refractive index is varied by the tissue fluid. Therefore, in the second step, the energy radiation may also be performed while the tissue liquid is removed or after the tissue fluid is removed. Further, in the second step, in place of the tissue fluid, liquid or gas as substitute for the tissue fluid, for example, physiological saline, may also be supplied.

### Other Example

In FIG. 5, the example in which the medicinal agent is directly injected into the tumor shown in the tumor image 24p in a state of the affected part illustrated in the ultrasound image of FIG. 4 is described. In other words, in the example of FIG. 5, the puncture needle 30 is directly stuck into the tumor and the medicinal agent is injected into the tumor. However, other than direct injection of the medicinal agent into the tumor, an effective treatment can be performed by injecting the medicinal agent to a peripheral site in some cases. For example, the cyst itself may become cancerous, and the cyst may simultaneously cause minute cancer in the pancreatic parenchyma. Therefore, direct injection of the medicinal agent into the cyst is also effective as treatment.

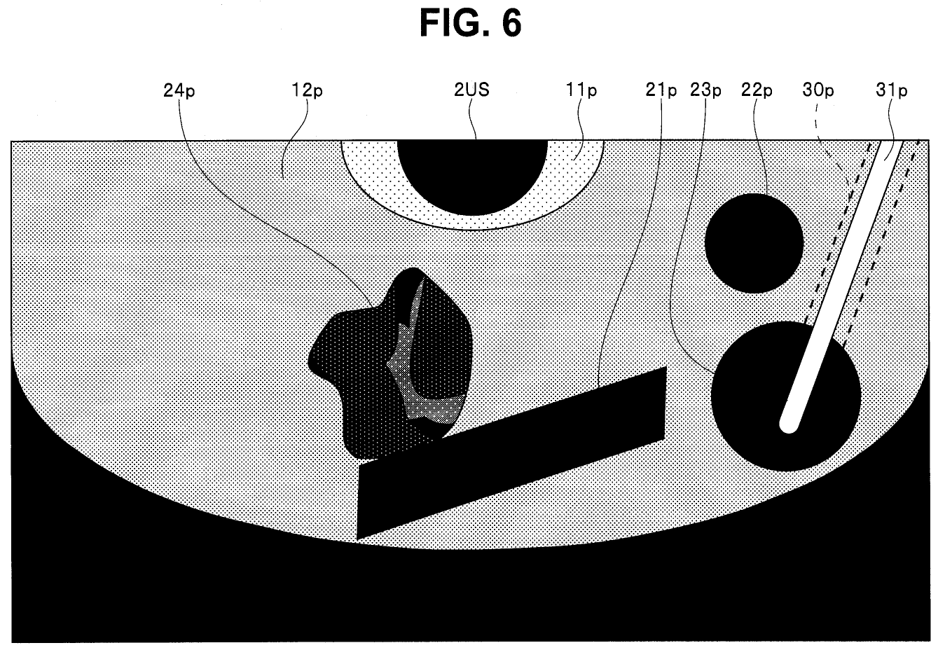

FIG. 6 is an explanatory diagram illustrating a state where the puncture needle is stuck into a pancreatic cyst. In an example of FIG. 6, the puncture needle 30 is stuck into the cyst shown in the cyst image 23p to inject the medicinal agent in the first step. Note that, in the first step, the energy radiation probe 7 is not inserted into the puncture needle 30. Thereafter, the energy radiation probe 7 is inserted into the needle tube of the puncture needle 30, the distal end of the energy radiation probe 7 is protruded from the distal end of the puncture needle 30 as shown in the probe distal-end image 31p, and the energy radiation probe 7 is placed on a position where the cyst shown in the cyst image 23p can be irradiated with the light from the energy radiation probe 7. Thereafter, the cyst is irradiated with the therapeutic light from the distal end of the energy radiation probe 7.

Further, in consideration of the fact that the pancreatic duct is a site where cancer frequently occurs, direct injection of the medicinal agent into the pancreatic duct is also effective.

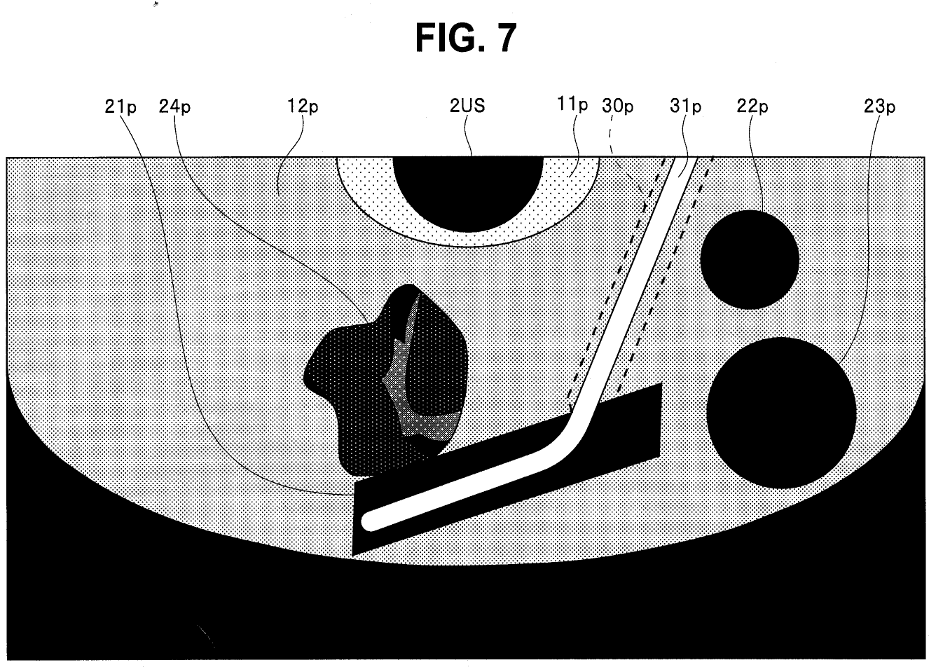

FIG. 7 is an explanatory diagram illustrating a state where the puncture needle is stuck into the pancreatic duct. In an example of FIG. 7, the puncture needle 30 is stuck into the pancreatic duct shown in the pancreatic duct image 21p to inject the medicinal agent in the first step. Note that, in the first step, the energy radiation probe 7 is not inserted into the puncture needle 30. Thereafter, the energy radiation probe 7 is inserted into the needle tube of the puncture needle 30, the distal end of the energy radiation probe 7 is protruded from the distal end of the puncture needle 30 as shown in the probe distal-end image 31p, and the energy radiation probe 7 is placed on a position where the pancreatic duct shown in the pancreatic duct image 21p can be irradiated with the light from the energy radiation probe 7. Thereafter, the pancreatic duct is irradiated with the therapeutic light from the distal end of the energy radiation probe 7.

As the treatment to the pancreatic cancer, resection is often performed. Although indirect findings of the cancer are often observed, it is difficult to determine whether to perform resection from the indirect findings. Therefore, the treatment may not be performed and the cancer may progress. In contrast, in the energy radiation treatment according to the present embodiment, an invasion degree is low, and aggressive treatment to the indirect findings of the cancer is easily selected. Accordingly, the energy radiation treatment according to the present embodiment to the pancreatic duct is an extremely effective treatment method.

Further, even when there is a possibility that the cancer is present in the pancreatic parenchyma, the cancer cell itself and the cyst may not be captured in the ultrasound image. In the case, direct injection of the medicinal agent into the pancreatic parenchyma is also effective. In the case, it is considered that the injected medicinal agent binds to the cancer.

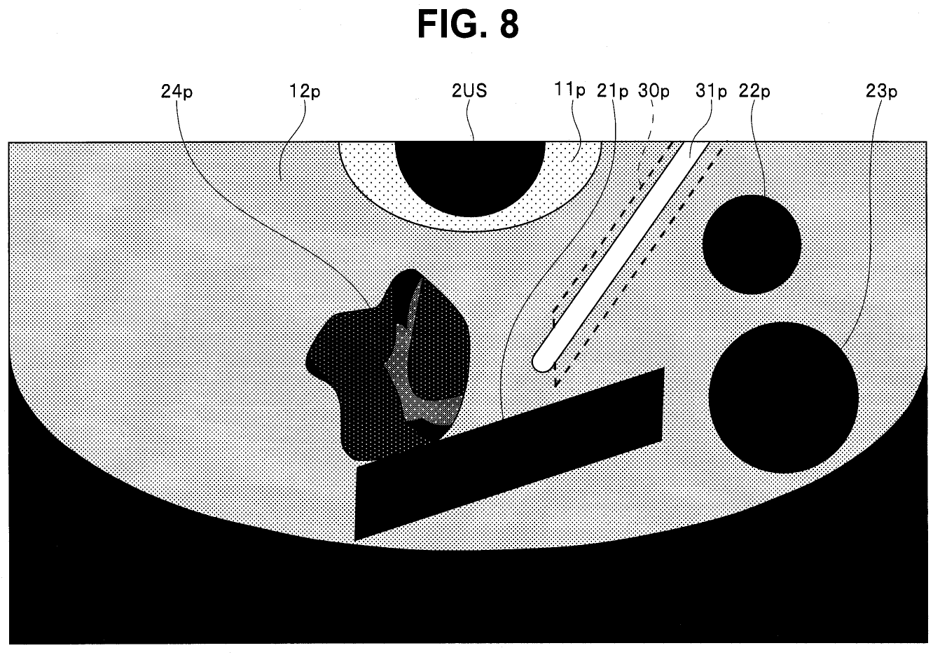

FIG. 8 is an explanatory diagram illustrating a state where the puncture needle is stuck into the pancreatic parenchyma. In an example of FIG. 8, the puncture needle 30 is stuck into the pancreatic parenchyma shown in the pancreas image 12p to inject the medicinal agent in the first step. Note that, in the first step, the energy radiation probe 7 is not inserted into the puncture needle 30. Thereafter, the energy radiation probe 7 is inserted into the needle tube of the puncture needle 30, the distal end of the energy radiation probe 7 is protruded from the distal end of the puncture needle 30 as shown in the probe distal-end image 31p, and the energy radiation probe 7 is placed on a position where the pancreatic parenchyma shown in the pancreas image 12p can be irradiated with the light from the energy radiation probe 7. Thereafter, the pancreatic parenchyma is irradiated with the therapeutic light from the distal end of the energy radiation probe 7. In the case, based on a range where the therapeutic light from the energy radiation probe 7 reaches, the injection of the medicinal agent and light radiation are performed a plurality of times while the position of the energy radiation probe 7 is changed.

In place of a method in which the fistula is formed in the target organ by the puncture needle 30 and the energy radiation probe 7 is inserted into the fistula as illustrated in FIGS. 5 to 8, the energy radiation probe 7 may be used in a method in which irradiation is performed from inside of the stomach or the duodenum. At this time, the energy radiation probe 7 may be used not through the treatment instrument channel 2ac of the ultrasound endoscope 2, but through a treatment instrument channel of another endoscope. Further, the energy radiation probe 7 may be directly inserted into the body.

When the light radiation device is provided at the distal end of the ultrasound endoscope 2, and light and ultrasound waves are emitted in the same direction in synchronization with each other, the ultrasound endoscope 2 can be used as a photoacoustic endoscope. Using the photoacoustic endoscope makes it possible to acquire a photoacoustic image of the target organ from the inside of the stomach or the duodenum, and to perform therapeutic light radiation to the medicinal agent at the same time. At this time, a wavelength, intensity, and a timing of the radiation light may be adjusted to form light optimized for the therapeutic light, and the light for treatment and light for acquisition of the photoacoustic image may be sequentially switched, thereby improving efficiency of the treatment.

The photoacoustic apparatus may not be an apparatus inserted into the body like the above-described photoacoustic endoscope, and may be an external photoacoustic apparatus.

Further, using the photoacoustic apparatus may enable monitoring of a consumption state of the medicinal agent in the second step. As described in the first step, the dispersion state of the medicinal agent may be seen from the photoacoustic image, which is similar to finding the fact that the medicinal agent reacts to the therapeutic light, acts on the cancer cells, and then disappears. Therefore, when the photoacoustic image is monitored, and it is determined to end the energy radiation based on the fact that the medicinal agent cannot be confirmed, it is possible to surely make the medicinal agent react to the energy radiation by irradiation once to complete the treatment, and to minimize the treatment time period.

Note that the light radiation in S5 may be performed only for a predetermined time period. A radiation condition such as the radiation time period of the therapeutic light may be determined based on a result of effect confirmation described below.

### Third Step

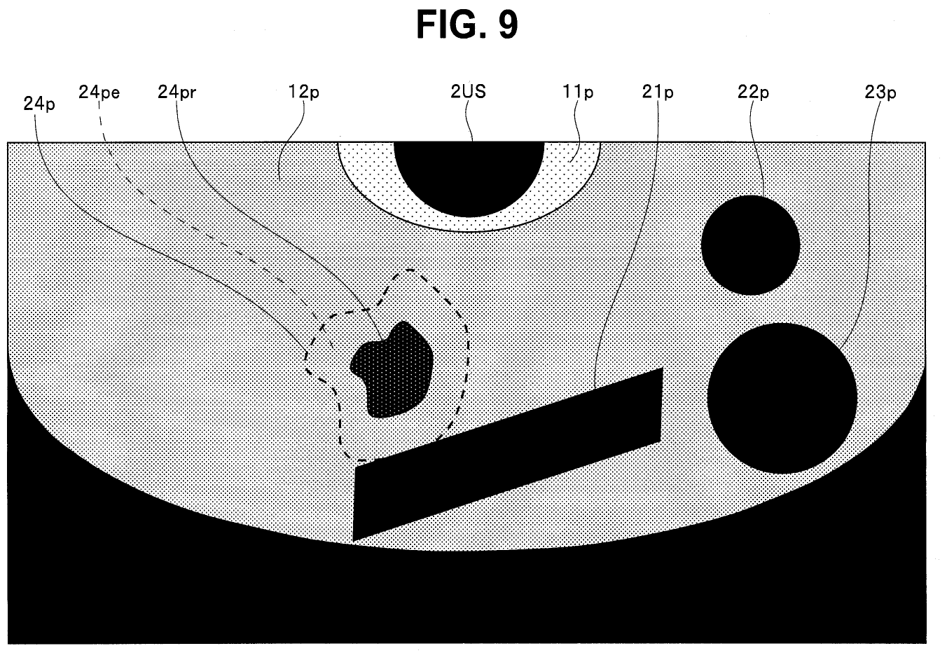

FIG. 9 to FIG. 11 are explanatory diagrams to explain the third step. FIG. 9 illustrates the ultrasound image to confirm effects in a case where the treatment by light radiation in the second step is performed in the state of FIG. 5.

FIG. 9 illustrates that the medicinal agent binding to the cancer has reacted by radiation of the therapeutic light in the second step, and as a result, a part of the cancer shown in the tumor image 24p in FIG. 5 becomes small by the therapeutic effects. A region illustrated by a dashed line in FIG. 9 indicates the tumor image 24p showing the cancer before the phototherapy, and a tumor image 24pr is an image showing the cancer remaining after radiation of the therapeutic light in the second step. A difference between the tumor image 24p and the tumor image 24pr is an image part 24pe showing a region where the cancer disappears by the phototherapy.

The doctor determines a state of the affected part, namely, therapeutic effects by the phototherapy with reference to the ultrasound images illustrated in FIG. 5 and FIG. 9 (S6). For example, the doctor confirms the therapeutic effects by before-and-after image comparison. The ultrasound image in a B mode (brightness mode) is a black/white gradation image. As a result of the treatment, the gradation (shades) of the ultrasound image is changed before and after the treatment. In FIG. 4 to FIG. 9, the change of the gradation is represented by change in type of hatching in consideration of visibility, but density of the hatching does not necessarily accurately represent the gradation of an actual image.

As described above, the doctor can confirm the therapeutic effects through comparison of the ultrasound image acquired before radiation of the therapeutic light, the ultrasound image acquired during radiation of the therapeutic light, the ultrasound image acquired immediately after end of radiation of the therapeutic light, and the ultrasound image acquired after a predetermined time period is elapsed from end of radiation of the therapeutic light.

The ultrasound observation apparatus 3 may include a control circuit 3a to automate various kinds of determinations using the ultrasound image in the first to third steps, for example, determination of the method of administering the medicinal agent, and confirmation of the therapeutic effects. The control circuit 3a may include a processor using a CPU (central processing unit), an FPGA (field programmable gate array), or the like. The control circuit 3a may control each of the units by performing operation based on programs stored in an unillustrated memory, or may realize a part or all of functions by an electronic circuit as hardware.

For example, the control circuit 3a can grasp a state of a patient by analyzing the acquired ultrasound image. For example, the control circuit 3a can determine a region of the tumor from luminance distribution of the ultrasound image, and can determine the therapeutic effects based on whether an area of the region of the tumor becomes less than or equal to a predetermined threshold.

When a result that the state of the affected part has reached an expected target state, for example, the cancer cells have been reduced by a predetermined threshold or more, is acquired through confirmation of the therapeutic effects, the doctor or the control circuit 3a may determine to end the treatment (S7). Further, as described above, the doctor or the control circuit 3a may determine to end the treatment based on the radiation time period of the therapeutic light.

Depending on the ultrasound image in the B mode, the therapeutic effects cannot be confirmed in some cases. In the case, the therapeutic effects may be confirmed using the ultrasound image using feature values of ultrasound reflected waves.

### Ultrasound Image in Mode Other Than B Mode

Various kinds of ultrasound images, for example, (1) an ultrasound image acquired using elastography, (2) an ultrasound image acquired using a frequency analysis technique, (3) an ultrasound image acquired using an ultrasound Doppler technique, (4) an ultrasound image acquired in a THE mode, (5) an ultrasound image acquired using an ultrasound contrast agent together, and (6) an ultrasound image acquired using a photoacoustic technique are usable for confirmation of the therapeutic effects.

(1) By the elastography, hard tissues and soft tissues in an organ can be identified, and an ultrasound image in which differences of the hardness are mapped in colors can be acquired. As a method of the elastography, there is a method of identifying the hard tissues and the soft tissues by using a fact that a distortion amount of tissue generated at the time of pressing operation of the ultrasound oscillator is different between the hard tissues and the soft tissues. Further, there is a method of identifying the hard tissues and the soft tissues by using a fact that a sound velocity of ultrasound shear waves passing through an inside of the tissues is different between the hard tissues and the soft tissues.

It is generally known that the tumor is hard as compared with surrounding tissues. It is considered that the hardness of the tumor is changed after radiation of the therapeutic light because of extinction of the cancer cells. In other words, direct findings whether the cancer has disappeared are obtained.

Further, it is considered that change such as inflammation occurs in tissues around the tumor due to influence by the medicinal agent and the therapeutic light. At the same time, it is considered that the hardness of the surrounding tissues is changed. Therefore, indirect findings whether the medicinal agent and the therapeutic light have been sufficiently supplied to the tumor are obtained.

It is considered that, in a case where the cancer cells are replaced with normal cells after extinction of the cancer cells, the hardness of the tissues is also changed. In other words, direct findings of the therapeutic effects are obtained.

(2) The frequency analysis technique is a technique performing coloration based on wavelengths of the ultrasound reflected waves received by the ultrasound endoscope 2. The ultrasound waves radiated from the ultrasound endoscope 2 are scattered by scatterers, and part of the scattered ultrasound waves are received as the reflected waves by the ultrasound endoscope 2. At this time, frequency spectra of the reflected waves are changed based on frequency components of the radiated ultrasound waves, and properties such as diameters, distribution, and density of the scatterers.

The frequency spectra acquired in such a manner are colored, which makes it possible to acquire the ultrasound image that is mapped in colors based on the properties of the scatterers in the tissues.

It is considered that, in a case where the cancer cells have gone extinct after radiation of the therapeutic light, change of a cell group is grasped as change in properties of the scatterers. In other words, direct findings whether the cancer has disappeared are obtained.

Further, it is considered that change such as inflammation occurs in tissues around the tumor due to influence by the medicinal agent and the therapeutic light. At the same time, it is considered that change of cells in the surrounding tissues is grasped as change in properties of the scatterers. Therefore, indirect findings whether the medicinal agent and the therapeutic light have been sufficiently supplied to the tumor are obtained.

It is considered that, in a case where the cancer cells are replaced with normal cells after extinction of the cancer cells, change of the cells in the tissues is also grasped as change in properties of the scatterers. In other words, direct findings of the therapeutic effects are obtained.

(3) The ultrasound Doppler technique uses Doppler effect. For example, the ultrasound reflected waves reflected by blood vessels are changed in frequency by the Doppler effect based on a direction and a flowing speed of blood. The frequency change generated in such a manner is colored, which makes it possible to acquire the ultrasound image in which the blood vessels are identified.

As described above, the blood vessels generally proliferate with proliferation of the cancer. In contrast, when the blood vessels are decreased with extinction of the cancer cells. In other words, when presence of the blood vessels is confirmed in the ultrasound image acquired by the ultrasound Doppler technique, indirect findings whether the cancer has disappeared are obtained.

(4) The THE (tissue harmonic echo) mode is a mode in which harmonic waves included in the ultrasound reflected waves are extracted by filtering, thereby acquiring the ultrasound image. A substance irradiated with ultrasound waves reflects the ultrasound waves on a surface, and generates harmonic waves as a result of expansion/contraction caused by reception of energy of the ultrasound waves. By using the harmonic waves, it is possible to acquire the ultrasound image up to a deep position with high definition as compared with a normal B-mode image.

(5) When a contrast agent for the ultrasound waves is injected into a vein and ultrasound waves optimized for the contrast agent are radiated, it is possible to cause the contrast agent to vibrate. The contrast agent itself can be drawn on an image by acquiring harmonic waves generated by the vibration as the ultrasound image. The contrast agent injected into the vein is carried to peripheral blood vessels. Therefore, it is possible to obtain indirect findings from presence/absence of proliferated blood vessels originating from the cancer, as in (3).

(6) To acquire a photoacoustic image as the ultrasound image, ultrasound waves and light (hereinafter, referred to as photoacoustic waves) are radiated. The light used to acquire the photoacoustic image may be intermittent light or light of a predetermined frequency. A substance receiving the light absorbs light energy and is thermally changed. When the heat is scattered, the substance returns to an original state, and expands and contracts to generate the ultrasound waves. As a result, the ultrasound image can be acquired. Further, a wavelength of the light to be radiated is controlled to an absorption band of target tissues such as cells and blood vessels, which makes it possible to obtain an ultrasound signal from the target tissues. In other words, when the photoacoustic images before and after radiation of the therapeutic light are compared, the direct findings about extinction of the cancer cells, the indirect findings about presence/absence of the blood vessels originating from the cancer cells, the indirect findings about reaching of the medicinal agent and the therapeutic light, and the direct findings about the therapeutic effects as described in (1) to (3) may be obtained.

Further, when the photoacoustic image is used, it is possible to confirm the injection state of the medicinal agent, and determination whether to terminate the injection of the medicinal agent can be easily performed. In other words, when the medicinal agent is irradiated with the photoacoustic waves, the medicinal agent may expand or contract. By generating a photoacoustic image based on the ultrasound waves generated with the expansion/contraction, the dispersion state of the medicinal agent indicating whether the medicinal agent has been absorbed to the cancer, namely, the accumulation degree of the medicinal agent on the affected part may be known.

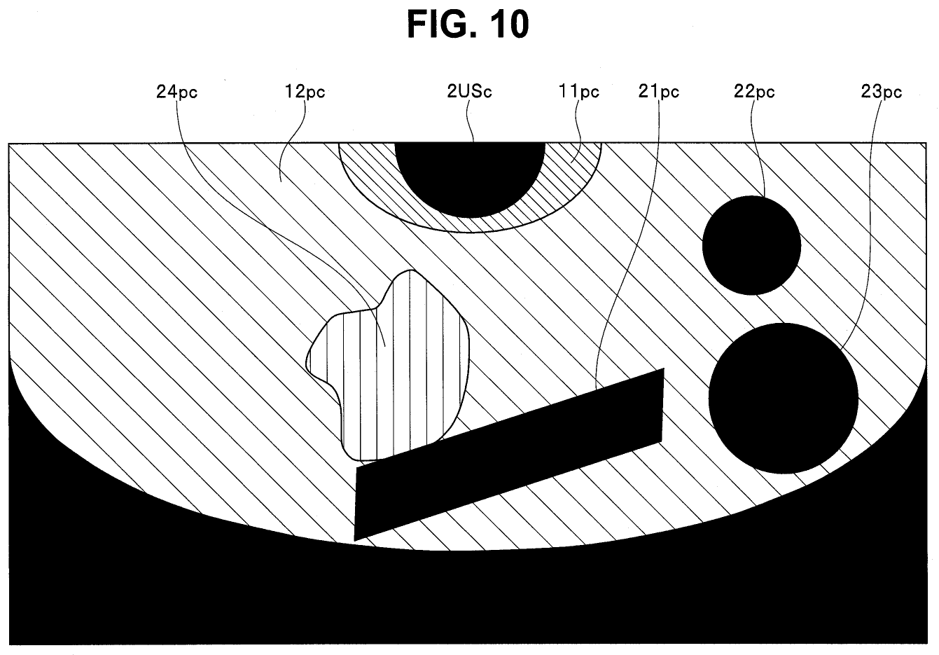

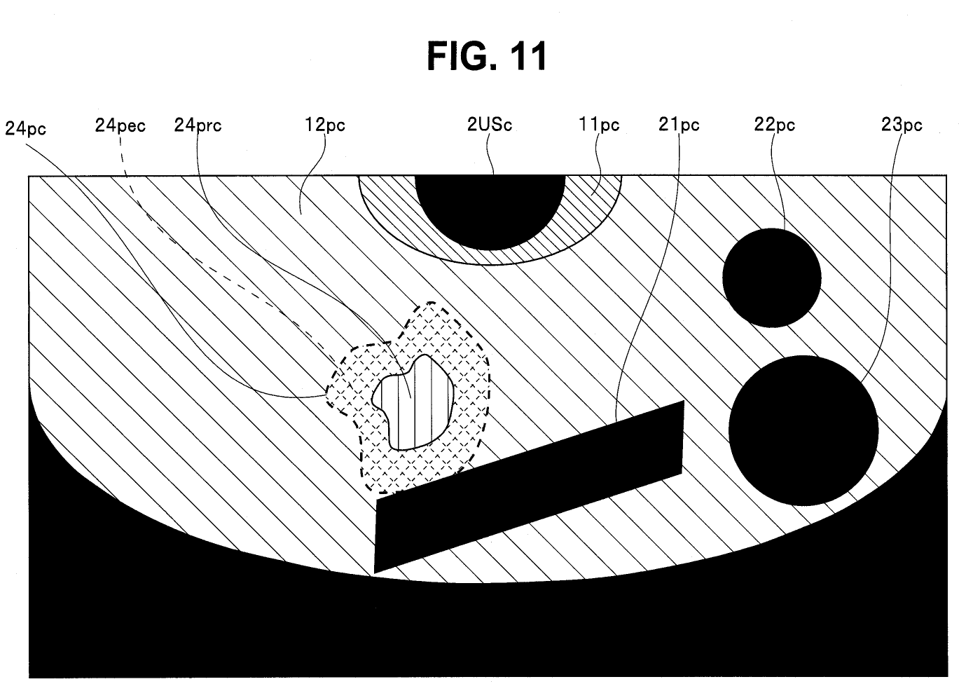

FIG. 10 is an explanatory diagram illustrating an image of the affected part in the ultrasound enhanced mode before radiation of the therapeutic light, and FIG. 11 is an explanatory diagram illustrating an image of the affected part in the ultrasound enhanced mode after radiation of the therapeutic light. In FIG. 10 and FIG. 11, portions different in hatching in each of the images indicate portions identified due to difference in color or black/white gradation in the ultrasound enhanced mode. FIG. 10 illustrates the state in FIG. 4 by the ultrasound image in the enhanced mode, and FIG. 11 illustrates the ultrasound image in the enhanced mode to confirm effects in a case where the treatment by light radiation in the second step is performed in the state of FIG. 10.

The image part 2US, the stomach wall image 11p, the pancreatic duct image 21p, the blood vessel image 22p, the cyst image 23p, the tumor image 24p, a treatment effective part 24pe, and the tumor remaining part 24pr in FIG. 4 and FIG. 9 are respectively acquired as an enhanced image part 2USc, an enhanced stomach wall image 11pc, an enhanced pancreatic duct image 21pc, an enhanced blood vessel image 22pc, an enhanced cyst image 23pc, an enhanced tumor image 24pc, an enhanced treatment effective part 24pec, and an enhanced tumor remaining part 24prc in FIG. 10 and FIG. 11.

The doctor confirms the therapeutic effects by before-and-after image comparison with reference to the enhanced ultrasound image illustrated in FIG. 10 and the enhanced ultrasound image illustrated in FIG. 11. Even in a case where change between the images before and after the treatment is small and confirmation of the therapeutic effects is not easy in the case of the black/white B-mode ultrasound image, change may appear between the images before and after the treatment in the case of the ultrasound images enhanced based on the feature values of the ultrasound reflected waves. It can be confirmed from the enhanced images that the tumor shown in the tumor image 24pc in FIG. 10 shrinks as shown in the tumor image 24prc in FIG. 11. The image part 24pec shows a part where the tumor shown in the original tumor image 24pc has been destroyed by the phototherapy. As described above, it is possible to confirm the therapeutic effects by using the ultrasound images displayed in the enhanced mode.

Likewise, the control circuit 3a can detect a size of the affected part, properties of the scatterers of the affected part, movement of blood in the affected part, increase/decrease of blood vessels in the affected part, expansion/contraction of the affected part, consumption of the medicinal agent, a reaching range of the medicinal agent and the therapeutic light, a healing state of the affected part and the surrounding tissues, and the like by image analysis on the ultrasound image, and can determine the condition such as the method of administering the medicinal agent and a period of the light radiation, from comparison with respective corresponding thresholds.

In the above-described various kinds of image comparison performed in order to confirm the therapeutic effects and the like, it is necessary to acquire the images to be compared, from the same cross-section. Therefore, an implant as a reference for the cross-section of the ultrasound image may be embedded. The ultrasound image is acquired (recorded) in a state where the implant is visually adjusted so as to be located at the same position in the same cross-section on the ultrasound image. Further, information on a position and inclination of each of the implant and the ultrasound endoscope 2 may be acquired by other sensors, and a position of the distal end of the ultrasound endoscope 2 may be adjusted based on the information outputted from the sensors.

In particular, in a case where the image is drawn by the pressing method in the elastography, a distortion amount of tissues is varied depending on a pressing amount. Therefore, colors are changed for each measurement even though distribution in the color map is the same. Thus, the pressing may be adjusted such that the colors are matched with colors of the implant in the ultrasound image to be compared. Alternatively, a numerical value may be corrected after the image is acquired.

### Acquisition Timing of Ultrasound Image

Extinction of the cancer cells by the phototherapy starts in response to radiation of the therapeutic light. However, even when radiation of the therapeutic light is stopped, extinction of the cancer cells progresses without stopping. The medicinal agent used for the treatment has immunoreactive potency to surrounding immune substances. In other words, even after the treatment, an effect of reduction of the cancer cells is obtained. Accordingly, even after the phototherapy ends, the state of the affected part may be confirmed by the above-described image comparison (S8), and it may be determined whether to end the treatment again (S9). Further, for example, there is a case where the therapeutic effects remarkably appear on and after the next day of the treatment rather than immediately after the phototherapy. Therefore, the light radiation in the second step may be repeated based on the endoscope image immediately after the treatment, and the light radiation in the second step may be newly performed based on the ultrasound image acquired later (S9 to S5).

In other words, as an acquisition timing of the ultrasound image used for confirmation of the therapeutic effects, for example, the following three patterns (a) to (c) are considered.

(a) Before treatment, and immediately after treatment (before first step or between first step and second step, and immediately after end of second step)
(b) Before treatment, and on and after next day of treatment (before first step or between first step and second step, and on and after next day after end of second step)
(C) Immediately after treatment, and on and after next day of treatment (immediately after end of second step, and on and after next day after end of second step)
The confirmation of the state of the affected part after the treatment includes confirmation about normal tissues around the lesion part and the like. The medicinal agent accumulates not only on the tumor but also on the normal tissues to some extent. It is possible to indirectly confirm the state of the tumor surrounded by the normal tissues by following up the state of the surrounding normal tissues. For example, in a case where the state of the affected part is confirmed a few days after the treatment, and recovery of the affected part is confirmed, it may be determined to end the treatment.

As described above, the third step may be simultaneously performed while the second step is performed, may be performed immediately after end of the second step, or may be performed after a predetermined time period is elapsed, for example, on the next day after end of the second step. Depending on a confirmation result of the effects in the third step, the treatment may return to the second step again, and radiation of the therapeutic light may be continued.

In a case of considering such a treatment method, the ultrasound endoscope 2 may be drawn out but the energy radiation probe 7 may be continuously placed. In other words, after the connection of the energy radiation probe 7 to the video processor with light source apparatus 5 or the light source apparatus 8 supplying the energy to the energy radiation probe 7 is disconnected, the ultrasound endoscope 2 is drawn out of the body in the state where the energy radiation probe 7 is placed. Further, after the energy radiation probe 7 is drawn out of the body through a nasal cavity, the energy radiation probe 7 is connected to the video processor with light source apparatus 5 or the light source apparatus 8 again. This makes it possible to perform the treatment by energy radiation at a desired timing while reducing a burden on the patient.

Note that, in the description of the above-described embodiment, the pancreatic cancer is described as an example. However, the energy radiation treatment method is similarly applicable to the energy radiation treatment for a parenchymal organ such as a liver and a gallbladder.

As described above, in the present embodiment, the ultrasound image is acquired in each of the first step including administration of the medicinal agent, the second step including placement of the energy radiation device and the energy radiation, and the third step including confirmation of the therapeutic effects, and in each of the treatment scenes, a course of the treatment is determined based on the ultrasound image. This makes it possible to rapidly perform administration of the medicinal agent, the energy radiation treatment, and confirmation of the therapeutic effects using the ultrasound endoscope.

### Second Embodiment

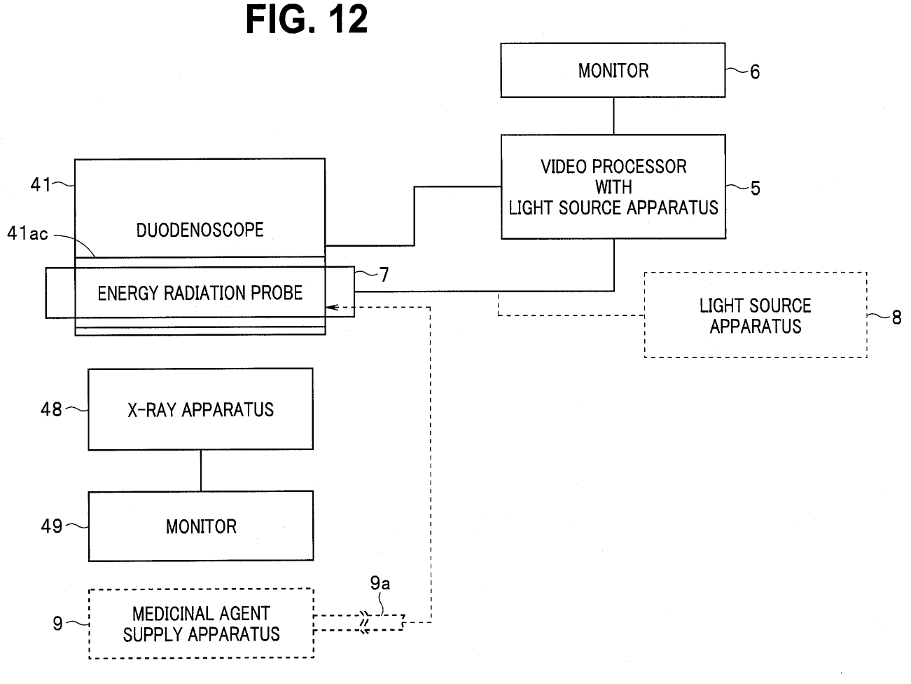

FIG. 12 to FIG. 15 relate to a second embodiment, and FIG. 12 is a block diagram illustrating the second embodiment. In FIG. 12, the components same as the components in FIG. 1 are denoted by the same reference numerals, and descriptions of the components are omitted.

In the present embodiment, an example in which the energy radiation treatment method is applied to treatment for cholangiocarcinoma or pancreatic ductal carcinoma is described. In the first embodiment, the distal end of the energy radiation probe 7 is brought close to the affected part by using the ultrasound image for the phototherapy of the pancreatic cancer, whereas in the second embodiment, the distal end of the energy radiation probe 7 is brought close to the affected part by using an optical image for treatment of cholangiocarcinoma or pancreatic ductal carcinoma. In other words, in the present embodiment, a duodenoscope is used in each of the first step including administration of the medicinal agent and the second step including placement of the energy radiation device and energy radiation, and the ultrasound image is used for confirmation of the therapeutic effects in the third step.

In the second embodiment, in place of the ultrasound endoscope 2 and the ultrasound observation apparatus 3, a duodenoscope 41 is adopted. Further, an X-ray apparatus 48, and a monitor 49 displaying an X-ray image acquired by the X-ray apparatus 48 are provided.

In FIG. 12, the duodenoscope 41 includes an elongated insertion portion 41a that has flexibility to be insertable into a body cavity, on a distal end side, and includes an unillustrated operation portion on a hand side. The video processor with light source apparatus 5, the light source apparatus 8, and the like are connected through a cable extending from the operation portion.

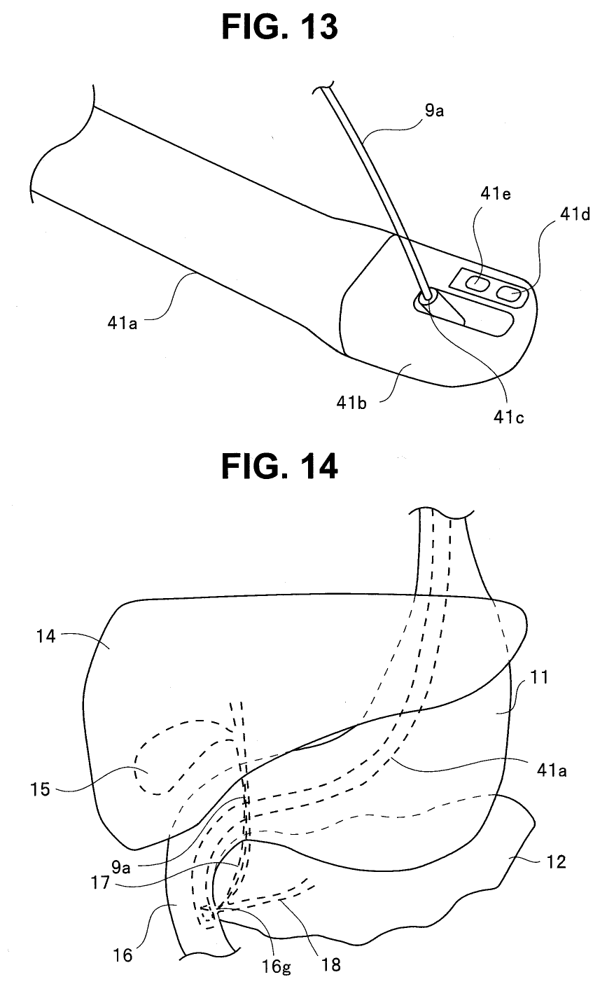

FIG. 13 is an explanatory diagram illustrating a schematic configuration of a distal end part of the insertion portion of the duodenoscope 41. A distal-end hard portion 41b including an unillustrated raising device is provided at a distal end of the insertion portion 41a of the duodenoscope 41. A distal end side of the insertion portion 41a is bendable by operation of the operation portion. The distal-end hard portion 41b is provided with an unillustrated image pickup sensor including an image pickup device such as a CCD. The duodenoscope 41 is supplied with illumination light from the video processor with light source apparatus 5, and irradiates a living body with the illumination light from an illumination window 41d. Reflected light from the living body is received by the image pickup device through an image pickup lens 41e, and is converted into an image pickup signal. The duodenoscope 41 is configured as a side-viewing endoscope including a viewing field in a direction intersecting a longitudinal axis of the insertion portion 41a. The image pickup signal from the image pickup device is supplied to the video processor with light source apparatus 5, and the video processor with light source apparatus 5 generates an endoscope image based on the image pickup signal. The video processor with light source apparatus 5 supplies the generated endoscope image to the monitor 6. As a result, the endoscope image based on the optical image is displayed on a screen of the monitor 6.

The insertion portion 41a of the duodenoscope 41 is provided with a treatment instrument channel 41ac, and the operation portion is provided with an opening portion communicating with the treatment instrument channel 41ac. A catheter 9a and an unillustrated guide wire can be inserted into the treatment instrument channel 41ac through the opening portion. The catheter 9a and the guide wire inserted into the treatment instrument channel 41ac can be protruded from a treatment instrument opening 41c of the distal-end hard portion 41b. A direction of the treatment instrument opening 41c can be changed to a direction intersecting the longitudinal axis of the insertion portion 41a by the raising device. This makes it possible to change a traveling direction of the catheter 9a, the guide wire, and the like protruding from the treatment instrument opening 41c to a predetermined direction.

### Treatment

### First Step

FIG. 14 is an explanatory diagram to explain procedures in the first and second steps using the duodenoscope 41.

The doctor inserts the catheter 9a into the treatment instrument channel 41ac of the duodenoscope 41. The doctor inserts the insertion portion 41a into the duodenum 16 while viewing the optical image acquired by the duodenoscope 41, and moves the distal-end hard portion 41b to a position facing a papilla 16g. The doctor pushes the catheter 9a into the treatment instrument channel 41ac while operating the raising device, and inserts a distal end of the catheter 9a into the papilla 16g. Further, the doctor operates the raising device while confirming a position of the distal end of the catheter 9a in the X-ray image acquired by the X-ray apparatus 48, injects an X-ray contrast agent from the catheter 9a as necessary, and inserts the distal end of the catheter 9a into the bile duct 17 or the pancreatic duct 18 from the papilla 16g. Note that FIG. 14 illustrates an example in which the catheter 9a is inserted into the bile duct 17. The doctor administers the medicinal agent to the affected part through the catheter 9a.

The other steps in the first step, namely, observation before administration of the medicinal agent and observation after administration of the medicinal agent are performed using the ultrasound image as in the first embodiment. In a case where the medicinal agent is a medicinal agent contrast-enhanced by the X-ray, the administration state of the medicinal agent can be observed in the X-ray image acquired by the X-ray apparatus 48.

### Second Step

The doctor inserts the unillustrated guide wire into the treatment instrument channel 41ac. Further, the doctor guides the guide wire to the affected part in a manner similar to guiding of the distal end of the catheter 9a to the affected part. The doctor places the energy radiation probe 7 on the affected part by using the guide wire, and performs energy radiation of the therapeutic light on the affected part.

The other steps in the second step, namely, observation during radiation of the therapeutic light and observation after radiation of the therapeutic light are performed using the ultrasound image as in the first embodiment.

### Third Step

The third step is performed using the ultrasound image as in the first embodiment.

As described above, in the present embodiment, administration of the medicinal agent, placement of the energy radiation device, and energy radiation are realized using the duodenoscope. In the present embodiment, it is also possible to use the ultrasound image for observation in each of the steps, and to perform rapid treatment. In the second embodiment, application to the bile duct is described. The treatment method is similarly applicable to the energy radiation treatment for a parenchymal organ such as a pancreas and a liver.

### Modification

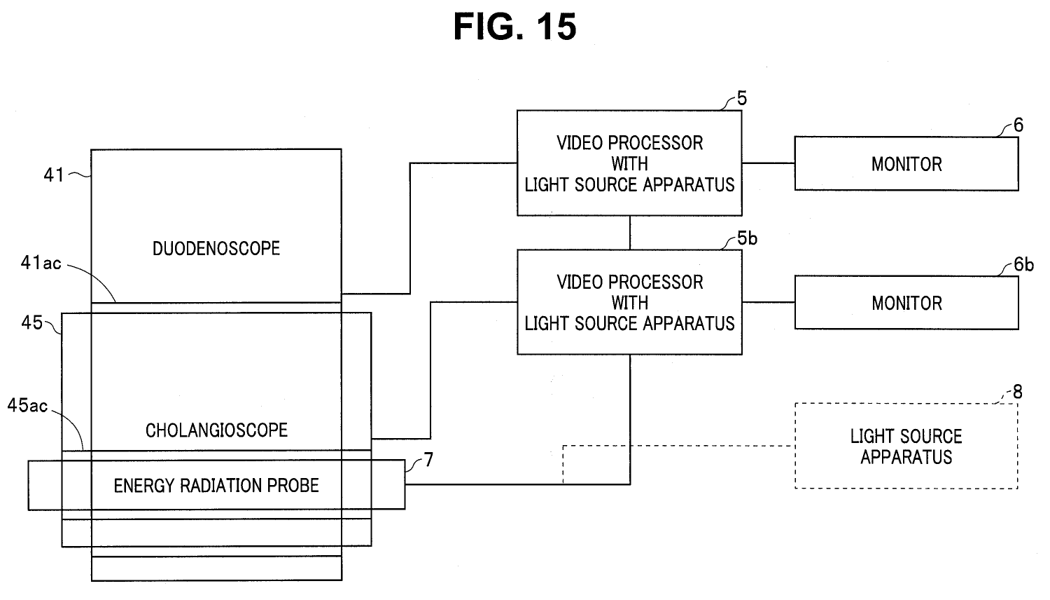

FIG. 15 is a block diagram illustrating a modification. In FIG. 15, the components same as the components in FIG. 12 are denoted by the same reference numerals, and descriptions of the components are omitted. In FIG. 15, illustrations of the medicinal agent supply apparatus 9, the X-ray apparatus 48, and the monitor 49 are omitted.

In the second embodiment, the first and second steps are performed using the duodenoscope 41. In the present modification, the first and second steps are performed using the duodenoscope 41 as a mother endoscope and a cholangioscope 45 as a baby endoscope.

In other words, in the case, the cholangioscope 45 is inserted into the treatment instrument channel 41ac of the duodenoscope 41, and an optical image is acquired by the cholangioscope 45. The doctor brings a distal end part of the cholangioscope 45 close to the papilla 16g while viewing the optical image acquired by the cholangioscope 45. Further, the doctor inserts the distal end part of the cholangioscope 45 into the bile duct 17 or the pancreatic duct 18 from the papilla 16g and brings the distal end part of the cholangioscope 45 close to the affected part while viewing the image by the cholangioscope 45. The doctor administers the medicinal agent through a treatment instrument channel 45ac of the cholangioscope 45 while viewing the optical image by the cholangioscope 45. The administration state of the medicinal agent is confirmed in the endoscope image by the cholangioscope 45.

The other steps in the first to third steps are similar to the above-described first and second embodiments.

### Third Embodiment

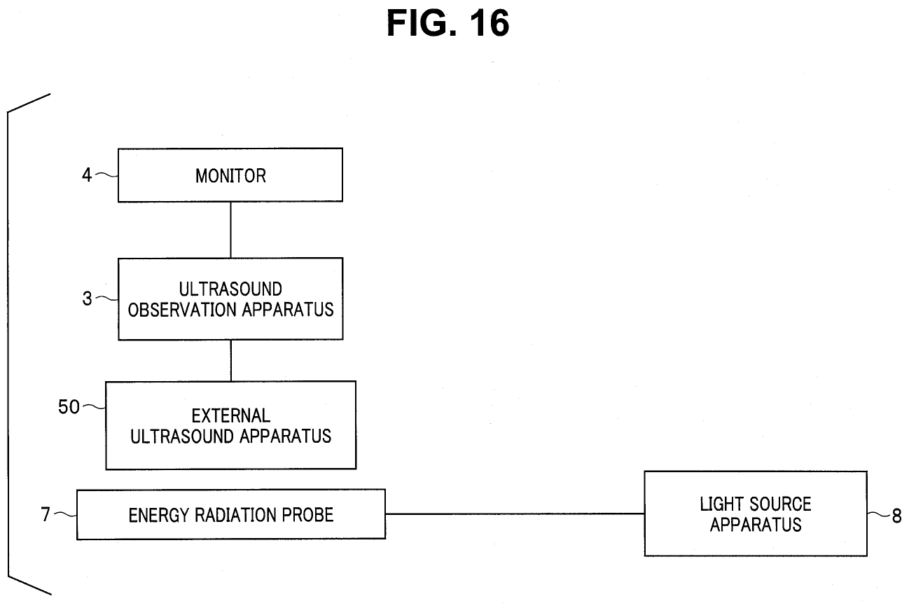

FIG. 16 is a block diagram illustrating a third embodiment. In FIG. 16, the components same as the components in FIG. 1 are denoted by the same reference numerals, and descriptions of the components are omitted.

The third embodiment illustrates an example in which the energy radiation treatment method is applied to treatment for liver cancer. In the third embodiment, treatment is performed from a body surface. In the third embodiment, the ultrasound image is used in the first step including administration of the medicinal agent, the second step including placement of the energy radiation device and energy radiation, and the third step including confirmation of the therapeutic effects.

In FIG. 16, an external ultrasound apparatus 50 has a configuration similar to the configuration of the ultrasound observation apparatus 3 in FIG. 1 except that the external ultrasound apparatus 50 is of an external type.

The third embodiment is different from the first embodiment in that the first to third steps are performed from outside of the body. The doctor holds an ultrasound oscillator of the external ultrasound apparatus 50 to a body surface, to cause the ultrasound image to be displayed. The doctor percutaneously confirms a position of the puncture needle, and places the distal end of the puncture needle in the affected part while viewing the ultrasound image displayed on the monitor 4. The doctor injects the medicinal agent into the needle tube of the puncture needle and administers the medicinal agent into the affected part while viewing the ultrasound image. The doctor causes the energy radiation probe 7 to irradiate the affected part into which the medicinal agent has been injected, with the therapeutic light, while viewing the ultrasound image. The doctor confirms the therapeutic effects by viewing the ultrasound image.

The other action and effects are similar to the first embodiment.

The embodiments are not limited to those described, and at the stage of practice, the present invention can be embodied by modifying the components without departing from the gist of the present invention. Further, various inventions can be formed by appropriately combining the plurality of components disclosed in the above-described embodiments. For example, some of all components described in each of the embodiments may be eliminated. Furthermore, the components in different embodiments may be appropriately combined.

What is claimed is:

1. An energy radiation treatment method comprising:
administering a medicinal agent to an affected part;
performing energy radiation with a predetermined energy on the affected part; and
confirming therapeutic effects by the energy radiation on the affected part, wherein
in the administering, a determination based on at least one ultrasound image based on ultrasound waves reflected from the affected part is performed.

2. The energy radiation treatment method according to claim 1, wherein
the administering includes obtaining affected part information about a state of the affected part based on the ultrasound image in at least one of a period before administration of the medicinal agent, a period during the administration of the medicinal agent, or a period after the administration of the medicinal agent, and
the medicinal agent is administered based on the affected part information.

3. The energy radiation treatment method according to claim 1, wherein the at least one ultrasound image includes an ultrasound image based on photoacoustic waves obtained by irradiating an inside of a living body with light and ultrasound waves.

4. The energy radiation treatment method according to claim 3, wherein the administering further includes observing injection of the medicinal agent in the ultrasound image based on the photoacoustic waves.

5. The energy radiation treatment method according to claim 2, wherein, in the obtaining of the affected part information, a method of administering the medicinal agent is determined based on at least one of a positional state of a lesion part, a state of stroma, or a state of a blood vessel.

6. The energy radiation treatment method according to claim 3, wherein the confirming further includes monitoring consumption of the medicinal agent in the at least one ultrasound image based on the photoacoustic waves while performing the energy radiation, and determining an end of the energy radiation based on the medicinal agent not being confirmed.

7. The energy radiation treatment method according to claim 1, wherein, in the determination based on the at least one ultrasound image, at least one of a size of the affected part, properties of scatterers of the affected part, movement of blood in the affected part, increase/decrease of blood vessels in the affected part, expansion/contraction of the affected part, consumption of the medicinal agent, a reaching range of the medicinal agent and therapeutic light, or a healing state of the affected part and surrounding tissues is determined based on the ultrasound image.

8. The energy radiation treatment method according to claim 1, further comprising an additional determination performed through comparison of the ultrasound image in plurality acquired at different timings.

9. The energy radiation treatment method according to claim 1, wherein the predetermined energy is one of light, ultrasound vibration, or a neutron beam.

10. The energy radiation treatment method according to claim 1, wherein
the administering and the performing are, or performing is, repeated based on a confirmation result in the third step,
in the confirming, comparison of the at least one ultrasound image acquired in a period before start of the administering or a period between the administering and the performing, with the at least one ultrasound image acquired in a first period after an end of the performing, comparison of the at least one ultrasound image acquired in the period before start of the administering or the period between the administering and the performing, with the at least one ultrasound image acquired in a second period after an end of the performing and after the first period, or comparison of the at least one ultrasound image acquired in the first period with the at least one ultrasound image acquired in the second period, is performed, and
in the performing, a condition of the energy radiation is determined based on a comparison result in the confirming.

11. The energy radiation treatment method according to claim 10, wherein the second period is a period on or after a next day of the first period.

12. A system supporting energy radiation treatment, the system comprising:
an ultrasound endoscope including a channel, the ultrasound endoscope being configured to acquire an ultrasound image of an affected part, the channel allowing insertion of an energy radiation probe to perform energy radiation with predetermined energy on the affected part and being used to administer a medicinal agent to the affected part; and
a processor comprising hardware, the processor being configured to determine a method of administering the medicinal agent to the affected part and a radiation condition of the energy radiation based on image comparison of the ultrasound image.

13. The system according to claim 12, wherein the processor is configured to determine the method of administering the medicinal agent and the radiation condition of the energy radiation by determining at least one of a size of the affected part or properties of scatterers of the affected part, based on the ultrasound image.

14. The system according to claim 12, wherein the processor is configured to determine the method of administering the medicinal agent and the radiation condition of the energy radiation by determining at least one of movement of blood in the affected part, increase/decrease of blood vessels in the affected part, or expansion/contraction of the affected part, based on the ultrasound image.

15. The system according to claim 12, wherein the processor is configured to determine the method of administering the medicinal agent and the radiation condition of the energy radiation by determining at least one of consumption of the medicinal agent, reaching range of the medicinal agent and therapeutic light, or a healing state of the affected part and surrounding tissues, based on the ultrasound image.
